(19) United States
(12) Patent Application Publication
(10) Pub. No.: US 2015/0204880 A1
(43) Pub. Date: Jul. 23, 2015

(54) Urinary Crystal Detection System and Method Thereof

(71) Applicant: NATIONAL YANG MING UNIVERSITY, Taipei (TW)

(72) Inventors: Hui-Hua Chiang, Taipei (TW); Yi-Chun Chiu, Taipei (TW); Chih-Chia Huang, Taipei (TW)

(21) Appl. No.: 14/455,395

(22) Filed: Aug. 8, 2014

(30) Foreign Application Priority Data
Jan. 20, 2014 (TW) 103101919

(51) Int. Cl. G01N 33/58

(52) U.S. Cl. CPC G01N 33/585

(57) ABSTRACT

The present invention provides an urinary crystal detection system, which comprises a crystal collecting unit and a crystal detecting unit, wherein the crystal collecting unit comprises a magnetic separating device. Further, the present invention provides an urinary crystal detection method, which uses magnetic particles to adsorb onto urinary crystals, then, provides a magnetic field to separate the urinary crystals and detect their constituent by the Raman signals of the urinary crystal.

### CROSS-REFERENCE TO RELATED APPLICATIONS

This Non-provisional application claims priority under 35 U.S.C. §119(a) on Patent Application No(s). 103101919 filed in Taiwan, Republic of China Jan. 20, 2014, the entire contents of which are hereby incorporated by reference.

### FIELD OF THE INVENTION

The invention relates to the urinary crystal detection system. In particular, the urinary crystal detection system used herein to distinguish the types of urinary calculus in patients.

### BACKGROUND OF THE INVENTION

Urinary calculus is a common disease. The main components of normal urine are crystals and substances. The crystals can be calcium oxalate, calcium phosphate, uric acid, uric acid saline, cystine, xanthine and so on. When the dissolved concentration of crystal is saturation in the urine, it might appear to super-saturation. Then the dissolved crystals will precipitate and result in urinary calculus occurrence.

Traditionally, the urine sediment is used to detect different morphology of sediments. The urine sediment is using an optical microscope to further determine the types of the urinary crystals. The urine sediment is easy to make erroneous judgment cause of stereo structure and irregular shape of the crystals, and different faces of crystals may appear different physical forms. Determination of crystal types always depends on the experiences of the examiner, therefore it has largely humane errors caused different examiner may have different result. Briefly, the irregularly shaped crystals cannot be accurately determined by its appearance alone.

Because of the traditional detecting method is using a microscope to determine the type of the crystals, it takes much time to search the location of the urinary crystals in patients with less amount of crystals. The traditional detecting method might ignore most of urinary crystals because they are translucent. Moreover, the urine includes many substances such as proteins, cells, bacteria, and so on, these substances may interfere the determination. The patients may miss the opportunity for early treatment.

Currently, the urinary calculus detection is conducted by imaging methods, such as X-ray imaging system, urinary system imaging, computed tomography, ultrasound, etc. In addition, the composition of the urinary calculus can not be determined before acquiring the urine calculus.

Clinically, the urinary calculi is easy to recurin patients. It is very important to know the composition of urinary calculus. Furthermore the characterization of urinary calculi is also important for therapy and prevention purpose.

The analysis methods for urinary calculi are numerous, including X-ray imaging, chemical analysis, X-ray diffraction, infrared spectroscopy microscopy, scanning electron microscopy, chromatographic automatic analysis, thermal stress analysis, thin film polarizers microscopy and so on. Currently, the urinary calculus can only be acquired through surgery or from the extracorporeal shock wave procedure. The types of urinary calculus can only be analyzed after acquiring the urinary calculus sample.

Recently, some researches have demonstrated that there is a strong correlation between the composition of the urinary calculus and the type of urinary crystal in the urinary calculus patients. Therefore, the successful and reliable identification of the type of urinary crystal in the patients having urinary calculus plays an important role in the treatment or therapy of urinary calculus.

So far, people still have the problems in detection and analysis of urinary crystals. The traditional urinary crystals detecting method (relying examiner's visual observation under the microscope) is inconvenient and may cause erroneous result, and cannot directly and accurately determine the type the crystals to make therapeutic decisions.

The above visual observation method has its limitation, more importantly, it still cannot accurately and efficiently determine the types of urinary crystal. The present invention can efficiently collect and accurately determine the types of urinary crystals.

### SUMMARY OF THE INVENTION

The present invention provides the urinary crystal detection, and analysis system. The urinary crystal detection system can collect and detect urinary crystals from patients' urine samples, and further determine the types of urinary crystals. Since the type of the urinary crystal is closely related to the type of the urinary calculus, so the patients can have different treatments according to different types of the urinary crystals.

The urinary crystal detection system includes a crystal collecting unit and a crystal detecting unit. The crystal collecting unit includes a magnetic separating device.

The crystal collecting unit is used to receive an urine sample which is added plurality of magnetic particles. The magnetic particles will adsorb the urinary crystals in the sample. The adsorbed magnetic particles will assist to collect and dispose on a carrier when the magnetic separation device produces a magnetic field. The crystal detecting unit receives the carrier and determines the type of the urinary crystals.

In an embodiment of the invention, the magnetic separating device can be a magnet or a magnetite, which is not limited herein. All devices can provide magnetic fields are the scope in this invention.

Further, in an embodiment of the invention, the urinary crystal detection system include a sample processing unit for pretreatment the urine sample, the crystal collecting unit for collecting the crystals by magnetic method, and the crystal detecting unit.

The above sample processing unit comprises a pH detecting device and pH processing device. The pH detecting device measures the pH value of the urine sample. The pH processing device performs acid or alkali treatment according to the detected pH value.

The crystal detecting unit can be any detector, such as a microscopy, a Raman spectroscopy, an infrared spectroscopy, a X-ray diffraction (XRD) or image analyzer, which is not limited herein. In a preferred embodiment, the crystal detecting unit is a Raman spectroscopy which can obtain optical Raman scattering signals from urinary crystal and further to determine the type of the urinary crystals. Moreover, the crystal detecting unit comprises a signal analyzing device which including a database, wherein the database use to receive the Raman signals to analyze and compare. The database contains all kinds of standard Raman signals of urinary crystal, as well as the urinary calculi correspond to the type.

Besides, the present invention also provides an urinary crystal detection method, the steps are as follows:

First, the urinary samples are provided. Second, a plurality of magnetic particles is added to the urine sample, and the magnetic particles absorb onto urinary crystals in the urine sample. Third, a magnetic field is provided to the urine sample. Then, the magnetic field attracts and collects the urinary crystals that are adsorbed by the magnetic particles. Finally, the types of urinary crystals are determined.

In an embodiment of the invention, before adding plurality of magnetic particles to the urine sample, the urine sample is performed by a pretreatment step. The pretreatment step comprise detecting the pH value of the urinary sample and performing acid or alkali processes according to the pH value. If the pH value of urinary sample is less than 7, the urinary sample is executed the acid processes, and if the pH value of urinary sample is more than 7, the urinary sample is executed the alkali processes. Both processes are used to separate the proteins, cells and etc. from urinary crystals. The acid processes using 5%˜15% acetic acid added to the urinary sample, the alkali processes using 5%˜15% NaOH added to the urinary sample and both of which is not limited herein.

In the urinary crystal detection system, the method used to detect the type of the urinary crystals comprises microscopy analysis, Raman spectroscopy, infrared spectroscopy, or X-ray diffraction, which is not limited herein. In a preferred embodiment, the Raman spectroscopy is used in the invention, and the Raman scattering signals are compared with the database to determine the type of the urinary crystals.

Moreover, the present invention also provides a magnetic particle containing a core, a protective layer and a functional group. The protective layer covers the core. The functional group is modified on the surface of the protective layer, and the functional group is PO₄³⁻, fluorescent molecules or the combination thereof.

In an embodiment of the invention, the components of the cores can be Fe, Co, Ni, FeO₂, Fe₂O₃ or Fe₃O₄. The components of the protective layers can be a surfactant, a polymer layer, PSMA, a co-polymer layer, a carbon/silica inorganic layer, collagen or gelatin. In a preferred embodiment, the component of the cores of the magnetic particles is Fe₃O₄ coated with gelatin as the protective layer, which is not limited herein. Furthermore, in a preferred embodiment, when the protective layer is the gelatin and the fluorescent molecule is FITC. Moreover, in a preferred embodiment, the size of the magnetic particles is 5-200 nm, which is also not limited herein.

In an embodiment, the component of the protective layer can be collagen or gelatin. The collagen or the gelatin contains most kinds of amino acid which can chemically and physically adsorb most kinds of urinary crystals. The collagen or the gelatin also contains the benzene ring structure which can physically adsorb the urinary crystals including uric acid. In specific type of urinary crystal, the functional group at the surface of the magnetic particles surface can be further modified. For example, the surfaces of the magnetic particles are modified by R—PO₄³⁻ molecules which can attract Ca- and Mg-based urinary crystals with Ca²⁺ or Mg²⁺.

In another embodiment, the protective layer of the magnetic particle can be PSMA and can be modified by PO₄³⁻, fluorescent molecules or the combination of the above. After adsorbing Ca-urinary crystals, the magnetic particles modified by fluorescent molecules can mark the Ca-urinary crystals. It is beneficial to quickly observe the location of the urinary crystals under the fluorescent microscope and it also can be used for automated detecting apparatuses. The automated detecting apparatuses can detect the urinary crystals via the fluorescent molecules. In a preferred embodiment, the fluorescent molecules are Pararosaniline hydrochloride (PAR), Crystal Violet (CV) or Fluorescein isothiocyanate (FITC).

The magnetic particles as tracer and collector described here can be applied to the urinary crystal detection system and method. According to the different urinary crystals, the magnetic particles can be modified by different functional groups, and these can be used selectively or combinatively in the urinary crystal detection system, which is not limited herein.

In sum, the urinary crystal detection system and method of the invention can efficiently collect and detect micro-crystals in the urine. The urine of urinary calculus patients generally contains crystals. Magnetic nanoparticles is a magnetic target and an external magnetic field can be provided to separate urinary crystals/micro stones from other substances in urine. Types of urinary crystals can be determined by its Raman signals. Early detection can be achieved for urinary calculus patients, and it can detect whether there are micro stones or crystals or not. For emergency cases, if the patient has urinary calculus symptoms, this invention can also provide real-time diagnostic efficacy.

The invention has the advantages in assisting clinical examination to determine the crystals that may be hard to determine from its shape under the microscope (the original approach). The invention is more accurate, more specific, and can assist clinicians to determine individual treatment according to different types of urinary calculus, which can be determined from the type of urinary crystals. It is also convenient to keep tracking and monitoring the patients after urinary stone surgery and to reduce the relapse rate.

### BRIEF DESCRIPTION OF THE DRAWINGS

FIG. 1 is a diagram of the urinary crystal detection system in the invention.

FIG. 2 is an embodiment diagram of the urinary crystal detection system in the invention.

FIG. 3A is a diagram of the magnetic particles in the invention.

FIG. 3B shows an embodiment diagram of the magnetic particles in the invention.

FIG. 3C shows a diagram of a third embodiment of the magnetic particle in the invention.

FIG. 3D shows a diagram of a fourth embodiment of the magnetic particle in the invention.

FIG. 4A shows an electron microscope diagram of the magnetic particles with the size of about 200 nm.

FIG. 4B shows an electron microscope diagram of the magnetic particles with the size of about 13 nm.

FIG. 5A is a diagram showing pure substance powder of Calcium oxalate.

FIG. 5B is a diagram showing the magnetic particles formed by Fe₃O₄ added surface-modified PO₄³⁻. The magnetic particles are adsorbed onto the Calcium oxalate in urine. FIG. 5C is a diagram showing the magnetic particles and the adsorbed Calcium oxalate attracted by an external magnet.

FIG. 6 shows a diagram of the magnetic particles adsorbed onto Calcium oxalate.

FIG. 7 shows a flow chart of the urinary crystal detection method.

FIG. 8A is a diagram of urinary crystals under the microscope, and the status of the urinary crystals is calcium oxalate dihydrate (COD). FIG. 8B is a diagram of Raman signals of the COD.

FIG. 9A is a diagram of urinary crystals under the microscope, and the status of the urinary crystals is hydroxyapatite (HAP).

FIG. 9B is a diagram of Raman signals of the HAP.

### DETAILED DESCRIPTION OF THE INVENTION

For clarity of disclosure, and not by way of limitation, the detailed description of the invention is divided into the subsections that follow.

The present invention provides an urinary crystal detection system. This urinary crystal detection system can collect and detect urinary crystals from patients' urine samples, so that different patients can have different treatments according to the types of the urinary calculus, which can be further determined from the type of the urinary crystal.

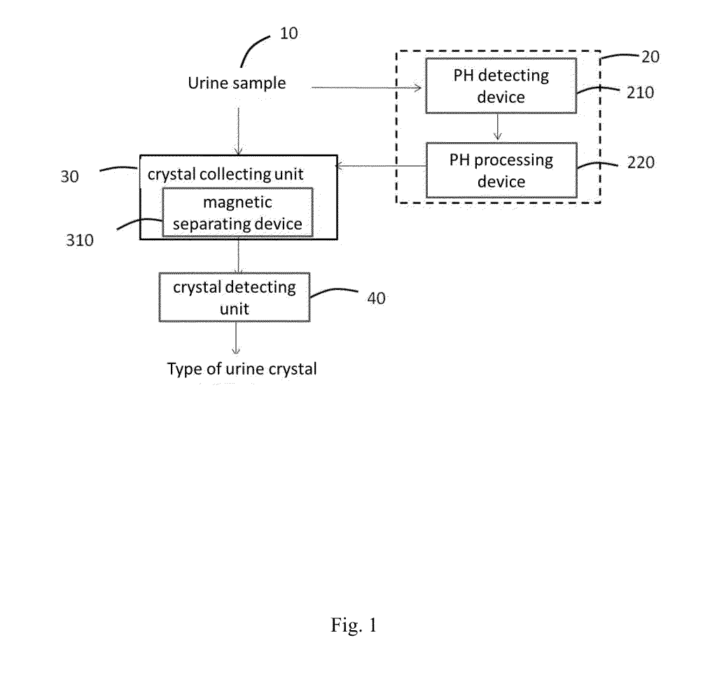

Please refer to FIG. 1 showing a diagram of the urinary crystal detection system in the invention. The urinary crystal detection system includes a crystal collecting unit 30 and a crystal detecting unit 40. Also, the crystal collecting unit 30 includes a magnetic separating device 310.

An urine sample 10 of a patient is received by the crystal collecting unit 30, and is added magnetic particles. The magnetic separating device 310 attracts the magnetic particles attached to the urinary crystals, and disposes the magnetic particles and transfers the urinary crystals to a carrier, such as Slides, quartz slides and so on. The crystal detecting unit 40 detects the type of the urinary crystal.

The crystal detecting unit 40 can be a microscopy, a Raman spectroscopy, an infrared spectroscopy, a X-ray diffraction (XRD) or image analyzer, which is not limited herein. In a preferred embodiment, the crystal detecting unit 40 is a Raman spectroscopy to measure Raman signals of the urinary crystals and further to determine the type of the urinary crystals.

The urinary crystal detection system also comprises a sample processing unit 20. The sample processing unit 20 including a pH detecting device 210 and pH processing device 220. The pH detecting device 210 measures the pH value of the urine sample 10. The urine sample 10 is detected by the pH detecting device 210 to generate a pH value, and executed processes by the pH processing device 220 according to the pH value. If the pH value of urine sample 10 is less than 7, the urine sample 10 is executed the acid processes; if the pH value of urine sample 10 is larger than 7, the urine sample 10 is executed the alkali processes. The acid processes and the alkali processes are used to separate the proteins, cells and etc. from the urinary crystals.

The urinary crystal detection system of the present invention can include or exclude the sample processing unit 20. The urine sample 10 can be directly used in the urinary crystal detection system of the present invention, or executed a pre-treatment by the sample processing unit 20 before the crystal collecting unit 30 receiving the urine sample 10. Moreover, a user can directly provide a pre-treated urine sample 10 for the urinary crystal detection system of the present invention, which is not limited herein.

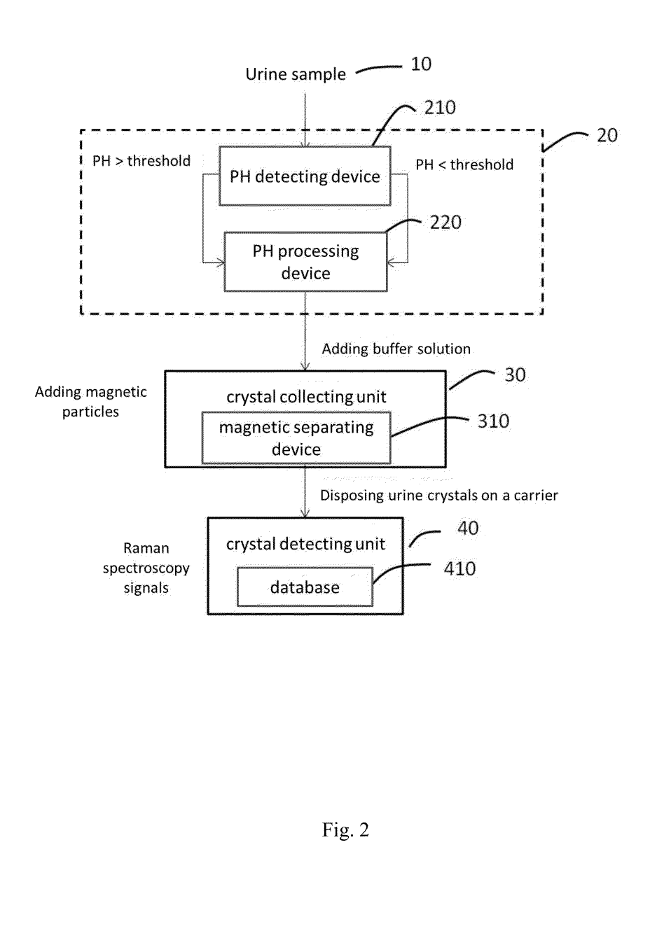

Please refer to FIG. 2 showing a diagram of another embodiment of the urinary crystal detection system in the invention. The urine sample 10 is detected by the pH detecting device 210 to generate a pH value, and executed processes according to the pH value. The amount of the urine sample 10 is 1-5 ml from a patient's urine, which is not limited herein.

In an embodiment, when the pH value of the urine sample 10 detected is larger than a threshold, the urine sample 10 is added alkali solution. For example, the pH value of the threshold pH=7, the pH value from urine sample 10 detected is larger than 7, the urine sample 10 is added 5%˜15% NaOH. In a preferred embodiment, the weight percentage concentration of NaOH is 10%, and the volume of NaOH is 200 μl, which is not limited herein.

In an embodiment, when the pH value from the urine sample 10 is lower than a threshold, the urine sample 10 is added acid solution. For example, the pH value of the threshold is 7, the pH value from the urine sample 10 is lower than 7, the urine sample 10 is added 5%˜15% acetic acid. In a preferred embodiment, the weight percentage concentration of acetic acid is 10%, and the volume of acetic acid is 200 μl, which is not limited herein.

The criteria of the pH value of the threshold can be adjusted by users. The value of pH=7 is an example in the invention, but not limited herein.

In an embodiment, after the urine sample 10 is executed above-mentioned acid or alkali processes, the urine sample 10 containing urinary crystals is added buffer solution, such as adding 5 ml of secondary water or three-times distilled water, to clean and remove impurities, which is not limited herein.

Then, the urinary crystals from urine sample 10 are received by the crystal collecting unit 30. In an embodiment, a plurality of magnetic particles are added to the urine sample 10, and the magnetic particles adsorb onto urinary crystals. The magnetic separating device 310 attracts the magnetic particles to collect the urinary crystals adsorbed by the magnetic particles. The collected magnetic particles and the urinary crystals are disposed on a carrier, such as slides, quartz slides and so on.

The magnetic separating device 310 disposes the urinary crystals on the carrier, such as quartz slides. The carrier is received by the crystal detecting unit 40 to execute detecting processes. The crystal detecting unit 40 can be any detector, such as a microscopy, a Raman spectroscopy, an infrared spectroscopy, a X-ray diffraction (XRD) or image analyzer, which is not limited herein. In a preferred embodiment, the crystal detecting unit 40 is a Raman spectroscopy to measure Raman signals from urinary crystals, which is not limited herein.

The crystal detecting unit 40 comprise a database 410. The database 410 is used to store the component information of all kinds of urinary crystal and urinary calculus. The urinary crystal is detected by the crystal detecting unit 40 to generate a detecting result. The detecting result is compared with the database 410 to determine the type of the urinary crystal. In a preferred embodiment, the crystal detecting unit 40 is a Raman spectroscopy to measure the Raman signals of the urinary crystals. The peak characteristics of Raman signals are compared with the component information of the database 410 and further to determine the type of the urinary crystals, which is not limited herein.

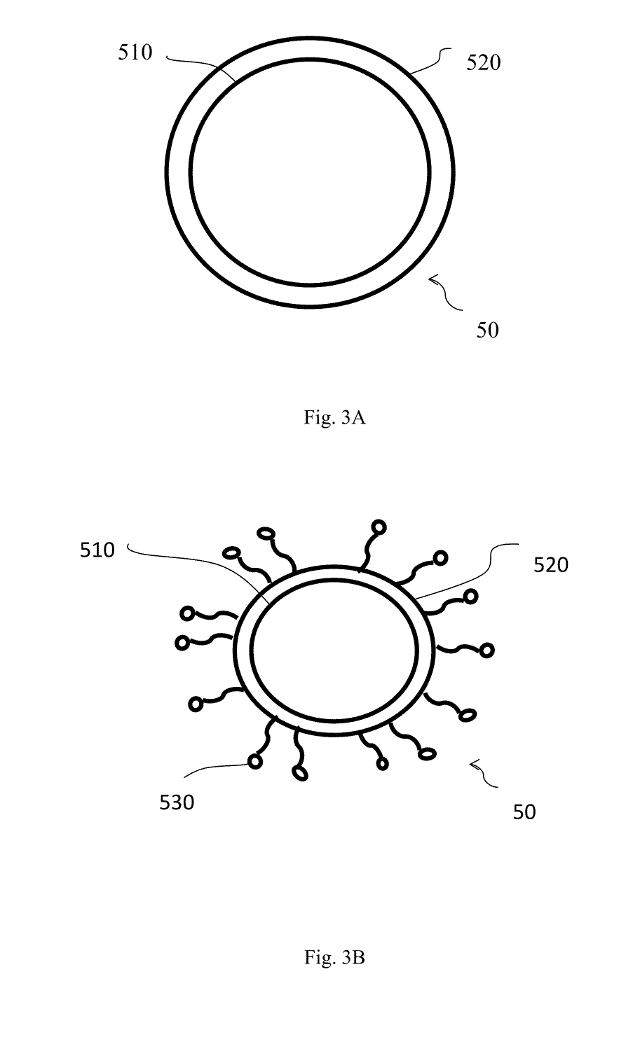

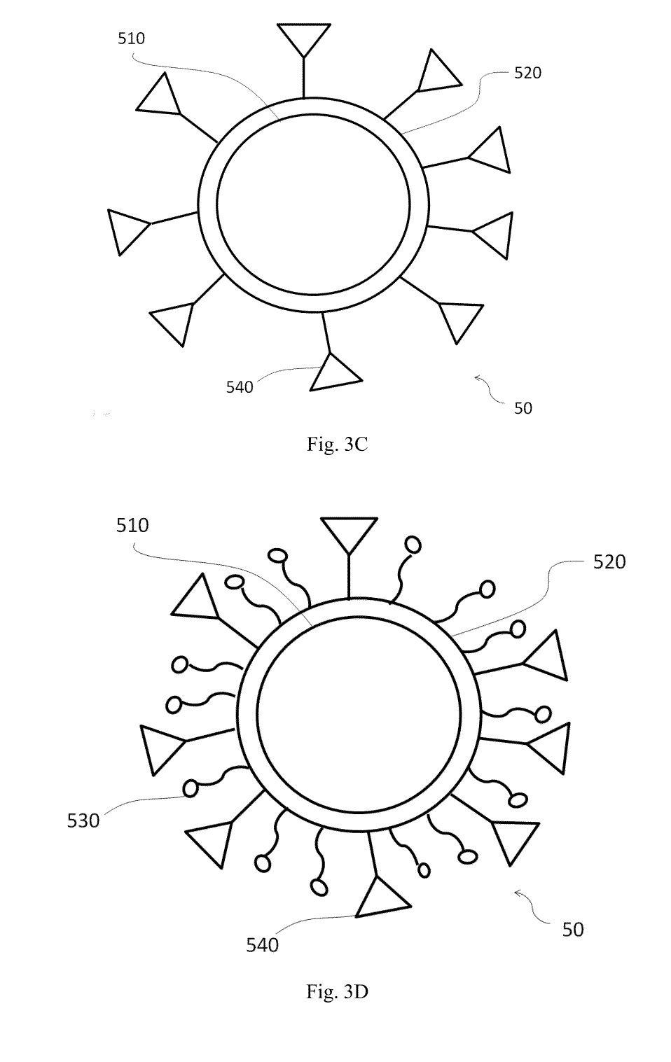

In an embodiment, the magnetic particles 50 are formed by a plurality of core 510 coated with protective layers 520. Please refer to FIG. 3A showing a diagram of the magnetic particle in the invention. The components of the cores 510 can be Fe, Co, Ni, FeO₂, Fe₂O₃ or Fe₃O₄, which is not limited herein. The components of the protective layers 520 can be a surfactant, a polymer layer, a carbon/silicon inorganic layer, collagen or gelatin. In a preferred embodiment, the component of the cores 510 of the magnetic particles 50 is Fe₃O₄ coated with gelatin as the protective layer 520. In a preferred embodiment, the component of the cores 510 of the magnetic particles 50 is Fe₃O₄ coated with PSMA as the protective layer 520, which is not limited herein.

The synthesis methods of the magnetic particles including coprecipitation method, thermal-chemical-vapor-deposition method, reduction method, micellar method, hydrothermal method and so on. Various synthetic methods are studied by scholars, which is not limited herein.

The main components of urinary calculi are calcium, magnesium, uric acid (such as calcium oxalate, magnesium ammonium phosphate). In a preferred embodiment, the magnetic particles 50 is Fe₃O₄ coated with gelatin (containing a benzene ring), and the component of the gelatin is animal collagen, which is a kind of natural high molecular polypeptide polymer. Therefore, the protective layer 520 composed of gelatin as magnetic particles 50 can adsorb most kinds of urinary crystals. In a situation of adsorbing a specific type of urinary crystal, the surface of the magnetic particles 50 can be further modified a functional group to make the magnetic particles 50 can select types of urinary crystal, such as shown in FIG. 3B.

In another embodiment of the invention, the component of the cores 510 of the magnetic particles 50 is Fe₃O₄ coated with PSMA or gelatin as the protective layer 520. The PSMA includes benzene ring structure to adsorb most kinds of organic urinary crystals, such as adsorbing uric acid crystals, but which is not limited herein.

In an embodiment of the invention, the magnetic particles 50 are formed by Fe₃O₄ and coated with surface-modified PO₄³⁻, as shown in FIG. 3B. The characteristic of R—PO₄³⁻ molecule is easy to bind with calcium ions or magnesium ions, thus the functional group of R—PO₄³⁻ have chemical bonding with the surfaces of magnetic nanoparticles of Fe₃O₄. The surfaces of nanoparticles with functional PO₄³⁻ group can further to form a covalent bond with calcium and magnesium. That is, the molecular structure is easy to have chemical reaction and bind with urine acid molecules, and it is effective to separate and purify urinary crystals with calcium or magnesium. Another example of magnetic particles 50 coated with the gelatin which structure contain benzene ring-rich functional group and hydrogen bond can be used for the adsorption of organic urinary crystals, such as adsorption of uric acid crystals, which is not limited herein.

In another embodiment, the surfaces of the magnetic particles 50 are modified by fluorescent molecules 540, such as Pararosaniline hydrochloride (PAR), Crystal Violet (CV) or Fluorescein isothiocyanate (FITC), as shown in FIG. 3C. After adsorbing urinary crystals, the magnetic particles 50 modified by fluorescent molecules 540 can mark the uric acid crystals. It is beneficial to quickly and accurately detect the types of the urinary crystals and it also can be used for automated detecting apparatuses. The automated detecting apparatuses can detect the urinary crystals via the fluorescent molecules. Any component having a benzene ring structure, NH₂ functional group or fluorescent molecules with positive charge can be used, the present invention is not limited thereto.

Furthermore, in an embodiment, in the FIG. 3D, the surfaces of the magnetic particles 50 can be modified by PO₄³⁻ 530 and fluorescent molecules 540 at the same time to make the magnetic particles 50 can have the functions of determining types of the urinary crystals and marking the urinary crystals.

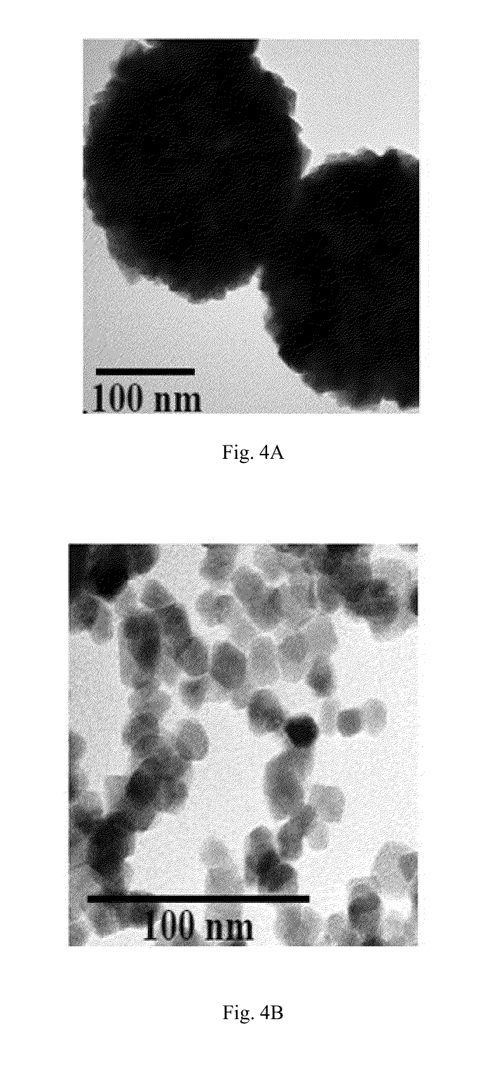

The size of magnetic particles is 5-200 nm, Please refers to FIG. 4A and FIG. 4B showing the electron microscope diagrams of different sizes of the magnetic particles. The size of magnetic particles in FIG. 4A is about 200 nm and the size of magnetic particles in FIG. 4B is about 13 nm. The preferred size of magnetic particle is 13 nm, which is not limited herein.

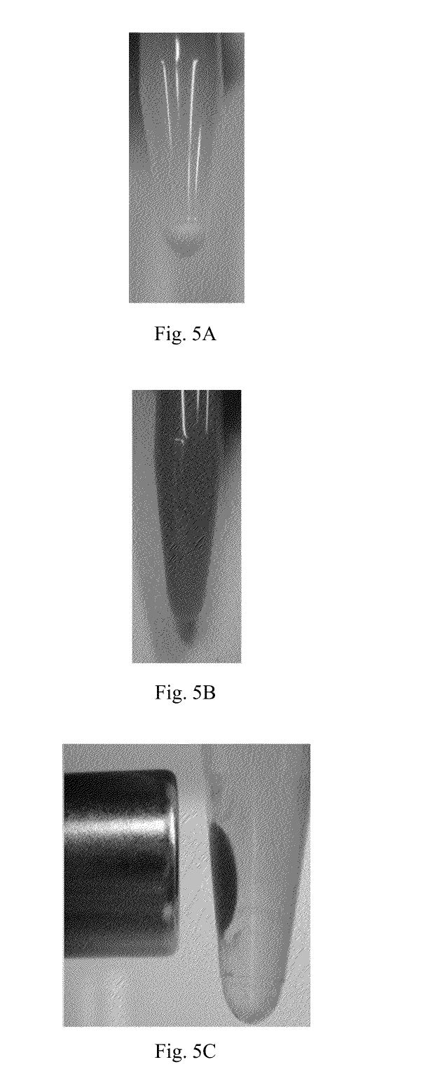

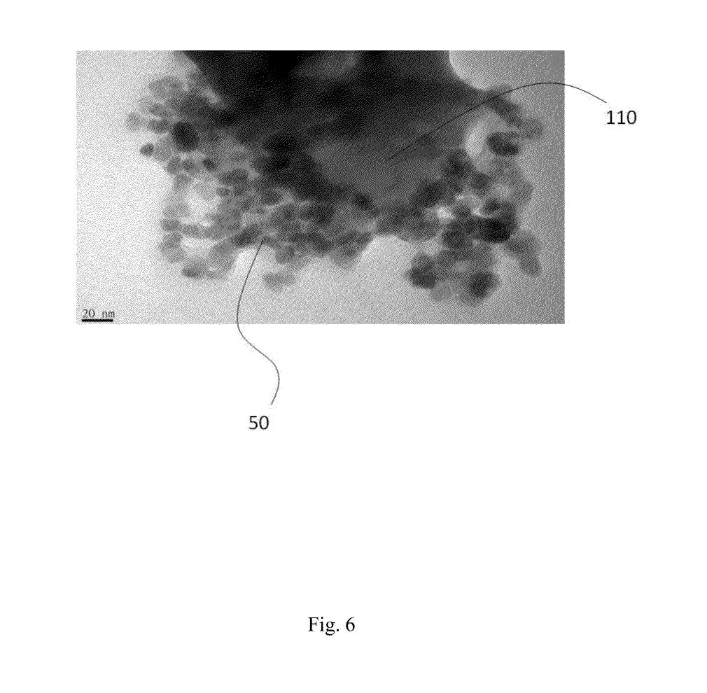

In above embodiment, please refer to FIG. 5A to FIG. 5C showing diagrams of surface-modified PO₄³⁻ surface-modified of Fe₃O₄ magnetic particles used to adsorb calcium oxalate. FIG. 5A is a diagram showing pure substance powder of Calcium oxalate. FIG. 5B is a diagram showing the magnetic particles formed by Fe₃O₄ added in surface-modified PO₄³⁻ groups. The magnetic particles adsorb onto the Calcium oxalate. FIG. 5C is a diagram showing the magnetic particles and the Calcium oxalate attracted by an external magnet. Please refer to FIG. 6 showing a diagram of the magnetic particles 50 adsorb onto Calcium oxalate 110. It means that if the urine sample is displayed with Calcium or Magnesium in urinary crystals, such as calcium oxalate, the urinary crystals can be separated by attaching to the magnetic particles 50 and adding an external magnetic field.

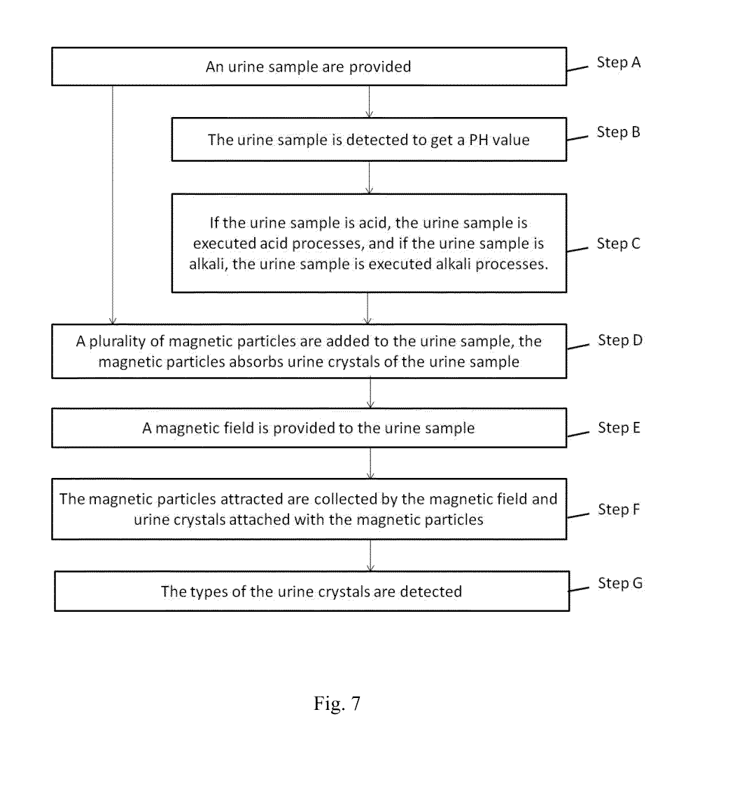

The present invention also provides an urinary crystal detection method. Please refer to FIG. 7 showing a flow chart of the urinary crystal detection method and the steps are as follows:

Step A. An urine sample are provided. In an embodiment, the amount of the urine sample is 1-5 ml from a patient's urine.

Step B. The urine sample is detected to get a pH value.

Step C. Processes are executed according to the pH value. If the pH value of urine sample 10 is less than 7, the urine sample is executed acid processes. And the pH value of urine sample 10 is larger than 7, the urine sample is executed alkali processes. In an embodiment, the acid processes comprise a step of adding 5%˜15% acetic acid to the urine sample, and the alkali processes comprise a step of adding 5%˜15% NaOH to the urine sample. After pretreatments, the urine sample forms urinary crystals, which is not limited herein.

Step D. A Plurality of magnetic particles are added to the urine sample, the magnetic particles adsorbs urinary crystals of the urine sample.

Step E. A magnetic field is provided to the urine sample. In an embodiment, the urine sample is provided an external magnet to attract the magnetic particles.

Step F. The magnetic particles attracted are collect by the magnetic field.

Step G. The types of the urinary crystals are detected. In an embodiment, the Raman signal of the urinary crystals are detected to get an analyzing result. The analyzing result is used to determine the type of the urinary crystals. In an embodiment, the Raman signals are compared with a database to determine the type of the urinary crystals.

The urinary crystal detection method can be executed step by step (above Step A to Step G orderly), and also be executed from step A and then directly performing step D. Each step can be adjusted according to different cases, which is not limited herein.

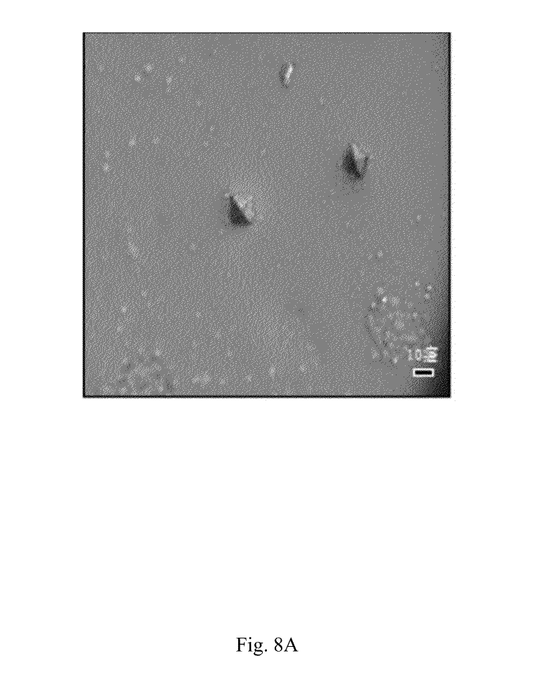

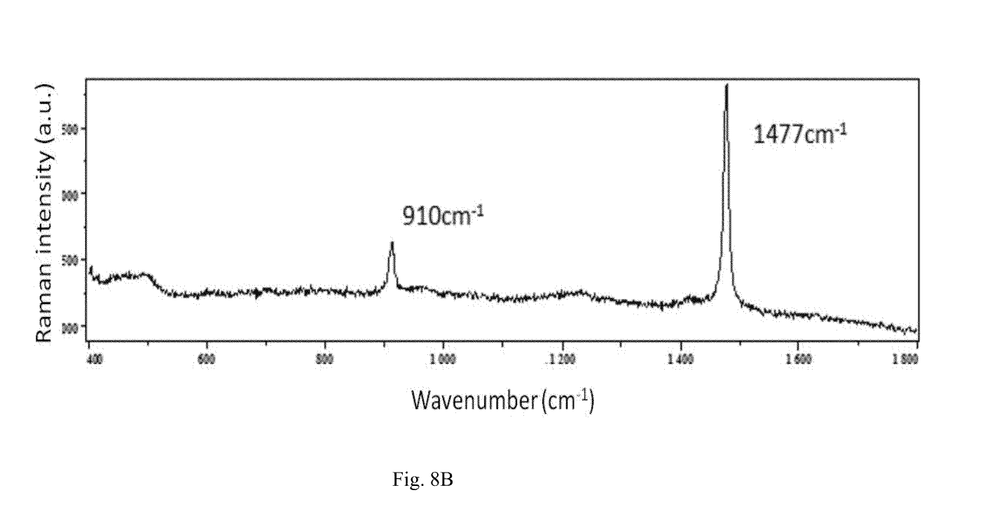

As above embodiment, please refer to FIG. 8A and FIG. 8B showing an urinary calculus patient's analyzing results from the urinary crystal detection system and method of the invention. FIG. 8A is a diagram of urinary crystals under a microscope, and the status of the urinary crystals is calcium oxalate dihydrate (COD). FIG. 8B is a diagram of Raman signals from COD. In FIG. 8B, it can show that the peak characteristics of Raman signals in COD are 910 (cm⁻¹) and 1477 (cm⁻¹).

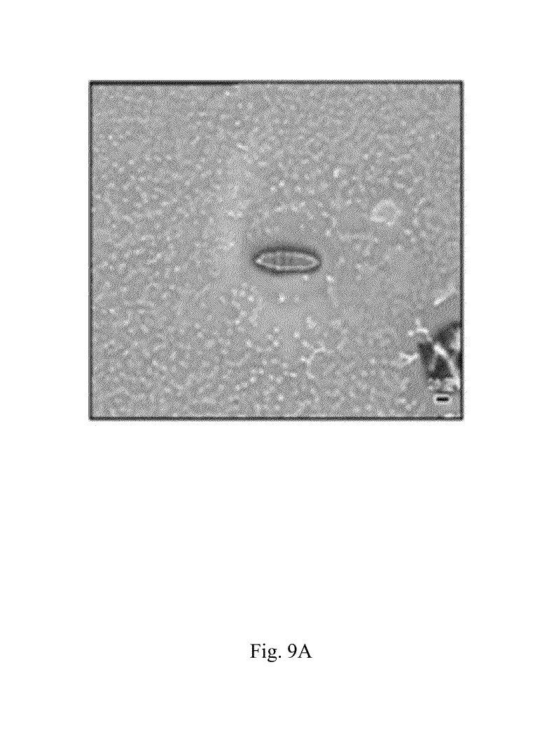

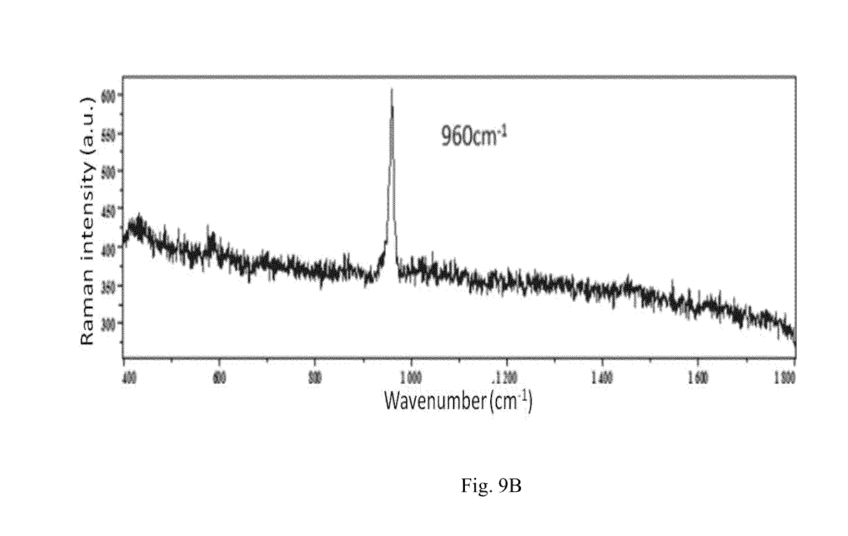

In another embodiment of the present invention, please refer to FIG. 9A and FIG. 9B showing another urinary calculus patient's analyzing results from the urinary crystal detection system and method of the invention. FIG. 9A is a diagram of urinary crystals under the microscope, and the status of the urinary crystals is hydroxyapatite (HAP). FIG. 9B is a diagram of Raman signals of the HAP. In FIG. 9B, it can show that the peak characteristic of Raman signals in HAP is 960 (cm⁻¹).

The urinary crystal detection system and method of the invention can be used to detect all kinds of urinary crystals, and construct a database via the analyzing results and further to be the reference for comparison afterwards. For example, in an embodiment of the invention, a database is constructed by Raman signals from all kinds of urinary crystals, as shown in table 1, but which is not limited herein.

TABLE 1
the database of Raman signals.

| | peak characteristics of |
|---|---|
| Urinary crystal/calculus | Raman signals (cm⁻¹) |
| COM (calcium oxalate monohydrate) | 895 \grave{ } 1462 |
| COD (calcium oxalate dihydrate) | 910 \grave{ } 1477 |
| DCPD (dicalcium phosphate dihydrate) | 986 |
| HAP(hydroxyapatite) | 960 |
| Uric acid | 623 \grave{ } 996 \grave{ } 1036 |
| Struvite (magnesium ammonium phosphate) | 563 \grave{ } 944 |
| Cystine | 498 |

In sum, the urinary crystal detection system and method of the invention can efficiently collect and detect the urinary crystals in the urine. The urine of an urinary calculus patient generally has urinary crystals. Custom made magnetic nanoparticles and an external magnetic field can be used to collect and separate urinary crystals/micro urine stones from other substances in urine. The types of urinary crystals can be determined by the Raman signals. Early detection can be achieved for urinary calculus patients by measuring their urinary crystal. For emergency cases, if the patient has urinary calculus symptoms, this invention can also provide real-time diagnostic efficacy. The invention has the advantages of assisting clinical examination to determine the type of the urinary crystals that may be hard to determine. It is more accurate, more specific, and can assist clinicians to provide individual treatment for the patients according to different types of urinary crystals. It is also convenient to keep tracking and monitoring the patients after the urine stone surgery and to reduce the relapse rate.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. An urinary crystal detection system, comprising:
a crystal collecting unit, receiving an urine sample and a plurality of magnetic particles are added to the urine sample, crystals of the urine sample, the magnetic particles attaching to urinary crystals of the urine sample, and the crystal collecting unit comprising:
a magnetic separating device, forming a magnetic field to attract the magnetic particles and further to collect the urinary crystals which are adsorbed by the magnetic particles, and the collected magnetic particles and the urinary crystals are disposed on a carrier; and
a crystal detecting unit, detecting the urinary crystals on the carrier and further to analyze the type of the urinary crystals.

2. The urinary crystal detection system of claim 1, wherein said magnetic particles are formed by a core and the core are coated with a protective layer.

3. The urinary crystal detection system of claim 2, wherein the components of the cores are Fe, Co, Ni, FeO₂, Fe₂O₃ or Fe₃O₄.

4. The urinary crystal detection system according to claim 2, wherein said the components of the protective layer are a surfactant, a polymer layer, a carbon/silicon inorganic layer, collagen or gelatin.

5. The urinary crystal detection system according to claim 4, wherein the polymer layer is Poly Styrene Methacrylic Acid (PSMA).

6. The urinary crystal detection system according to claim 5, wherein the surfaces of the magnetic particles are modified by fluorescent molecules.

7. The urinary crystal detection system according to claim 6, wherein the fluorescent molecules are Pararosaniline hydrochloride (PAR) and Crystal Violet (CV).

8. The urinary crystal detection system according to claim 4, wherein when the protective layer is the gelatin and then the gelatin is modified by the fluorescent molecule FITC.

9. The urinary crystal detection system of claim 2, wherein the component of the cores is Fe₃O₄ and the component of the protective layers is gelatin.

10. The urinary crystal detection system of claim 2, wherein said the surfaces of the magnetic particles are modified by PO₄³⁻ groups.

11. The urinary crystal detection system of claim 1, wherein said the sizes of the magnetic particle are 5 nm˜200 nm.

12. The urinary crystal detection system of claim 1, wherein the magnetic separating device is a magnet or a magnetite.

13. The urinary crystal detection system of claim 1, further comprising a sample processing unit, used to process the urine sample before the crystal collecting unit receives the urine sample.

14. The urinary crystal detection system of claim 13, the sample processing unit comprises:
a pH detecting device, detecting the pH value of urine sample; and
a pH processing device, executing the acid processes or the alkali processes according to the pH value.

15. The urinary crystal detection system of claim 1, wherein the crystal detecting unit is a microscopy, a Raman spectroscopy, an infrared spectroscopy, a X-ray diffraction (XRD).

16. The urinary crystal detection system of claim 15, wherein the crystal detecting unit is a Raman system to measure the Raman signals of the urinary crystals.

17. The urinary crystal detection system of claim 16, wherein the crystal detecting unit further comprises a signal analyzing device, used to analyze the type of the urinary crystals.

18. The urinary crystal detection system of claim 17, wherein the signal analyzing device comprising a database.

19. An urinary crystal detection method, comprising:
providing an urine sample;
a plurality of magnetic particles are added to the urine sample, the magnetic particles absorbing urinary crystals of the urine sample;
a magnetic field is provided to the urine sample;
collecting the magnetic particles attracted by the magnetic field and urinary crystals attached by the magnetic particles; and
detecting the type of the urinary crystals.

20. The urinary crystal detection method of claim 19, further comprises a step to process the urine sample before adding the magnetic particles to the urine sample.

21. The urinary crystal detection method of claim 20, wherein the step of processing the urine sample comprise:
detecting the urine sample to get a pH value; and
executing processes according to the pH value, if the pH value of urine sample is less than 7, the urine sample is executed the acid processes; if the pH value of urine sample 10 is larger than 7, the urine sample is executed the alkali processes.

22. The urinary crystal detection method of claim 21, wherein the acid processes comprise a step of adding 5%˜15% acetic acid to the urine sample, and the alkali processes comprise a step of adding 5%˜15% NaOH to the urine sample.

23. The urinary crystal detection method of claim 19, wherein the surfaces of the magnetic particles are modified by PO₄³⁻ to attach the urinary crystals with Ca or Mg.

24. The urinary crystal detection method of claim 19, wherein the component of the magnetic particles is Fe₃O₄ coated with gelatin.

25. The urinary crystal detection method according to claim 19, wherein the magnetic particles are formed by Fe₃O₄ coated with PSMA.

26. The urinary crystal detection method according to claim 25, wherein the surfaces of the magnetic particles are modified by fluorescent molecules.

27. The urinary crystal detection method according to claim 26, wherein the fluorescent molecules are PAR or CV.

28. The urinary crystal detection method according to claim 24, wherein the gelatin are modified by FITC.

29. The urinary crystal detection method according to claim 19, wherein the step of detecting the type of the urinary crystals comprises analyzing methods of microscopy, Raman spectroscopy, infrared spectroscopy, or X-ray diffraction.

30. The urinary crystal detection method according to claim 29, wherein the step of detecting the type of the urinary crystals comprises the analyzing method of Raman spectroscopy to detect Raman signals of the urinary crystals.

31. The urinary crystal detection method according to claim 30, further comprises a step of comparing the Raman signals with a database to determine the type of the urinary crystals.

32. A magnetic particle, comprising:
a core;
a protective layer, covering the core; and
a functional group, modified on the surface of the protective layer, and the functional group is PO₄³⁻, a fluorescent molecule or the combination thereof.

33. The magnetic particle according to claim 32, wherein the components of the cores are Fe, Co, Ni, FeO₂, Fe₂O₃ or Fe₃O₄.

34. The magnetic particle according to claim 32, wherein the components of the protective layer is a surfactant, a polymer layer, a carbon/silicon inorganic layer, collagen or gelatin.

35. The magnetic particle according to claim 34, wherein the polymer layer is PSMA.

36. The magnetic particle according to claim 32, wherein the fluorescent molecule is PAR or CV.

37. The magnetic particle according to claim 32, wherein the protective layer is the gelatin and the fluorescent molecule is FITC.

38. The magnetic particle according to claim 32, wherein size of the magnetic particle is 5 nm˜200 nm.
